(19) United States
(12) Patent Application Publication
(10) Pub. No.: US 2007/0264224 A1
(43) Pub. Date: Nov. 15, 2007

(54) Exfoliating and Softening Composition

(75) Inventors: Paul Morris, Flintshire (GB); Wayne Walton, Cheshire (GB)

Correspondence Address: DENNISON, SCHULTZ & MACDONALD, 1727 KING STREET, SUITE 105, ALEXANDRIA, VA, 22314, US

(73) Assignee: GELICITY (UK) LIMITED, SALTNEY, CHESHIRE (GB)

(21) Appl. No.: 11/663,577

(22) PCT Filed: Sep. 23, 2005
(86) PCT No.: PCT/GB05/03674

§ 371 Date: Mar. 23, 2007

(30) Foreign Application Priority Data
Sep. 24, 2004 (GB) 0421286.6
Jul. 5, 2005 (GB) 0513704.7

(51) Int. Cl. A61K 31/74; A61K 33/00; A61K 36/00; A61K 36/899

(52) U.S. Cl. 424/78.03; 424/724; 424/725; 424/750

(57) ABSTRACT

A skin exfoliating and softening composition, comprising sodium polyacrylate powder at between 5% to 70% by weight dispersed in a solution of base oil. The composition comprises sodium polyacrylate powder mixed with any suitable base oil including food grade oils, synthetic oils, natural oils or mineral oils, with added fragrances, anti-settling agents, aromatherapy oils and/or dyes. A method of exfoliating and softening skin is also disclosed as the composition can be used on any part of the body by rubbing it over the skin to produce an exfoliating and softening effect. After rubbing the composition on the skin for a short period of time, the treated area is then placed under running water or sprayed using an appropriate atomiser which causes the sodium polyacrylate powder to gel up and help the base oil and moisture to be absorbed by the skin. The invention has exfoliating properties by firstly removing dead skin cells from the surface of the skin and deep cleansing properties by causing the skin pores to open and sucking out moisture from the skin taking with it dirt etc. This allows the base oils and cleaner moisture to absorbed back into the skin leaving it feeling clean, smooth and soft. A method of manufacturing a skin exfoliating and softening composition is also disclosed.

The present invention relates to compositions that are useful for exfoliating and softening skin. In particular, the present invention relates to compositions that are useful for exfoliating and softening skin containing sodium polyacrylate powder as an active ingredient or agent dispersed in a solution of base oil.

Skin exfoliating products are well known and serve to remove dead skin cells from the surface of the skin. Generally, such exfoliating products comprise abrasive particles dispersed a cleansing solution, such as body washes and liquid hand soaps. These abrasive particles may be natural, such as walnut shells or grit or sand, or synthetic particles, such as polyethylene beads. After application of such exfoliating products, a separate skin moisturiser is often required to replace some of the natural oils that are lost from the surface of the skin during exfoliation.

The problem associated with many commercially available skin exfoliating products is that they can be sometimes too harsh for the skin; stripping the skin of its essential natural oils and requiring the separate application of a moisturiser.

It is the object of the present invention to provide compositions that are useful for exfoliating and softening skin and which are particularly suitable for cosmetic, toiletry, spa treatments and beauty products. In use, a small quantity of the composition is applied to the skin to produce an exfoliating and smoothing effect. When the skin is then rinsed in water, the sodium polyacrylate powder swells and turns to a soft gel which allows the base oil to be released into the skin which softens and moisturises the skin. The base oil and other additives soften and moisturise even the roughest of skin types and produces a soft exfoliating feeling on the skin. The present invention therefore provides compositions that both exfoliate and soften the skin in a single treatment. In a further object of the present invention, the composition opens and has a cleansing effect on skin pores.

According to the present invention there is provided a skin exfoliating and softening composition, comprising sodium polyacrylate powder at between 5% to 70% by weight dispersed in a solution of base oil.

Preferably, said skin exfoliating and softening composition comprises sodium polyacrylate powder at between 20% to 55% by weight. In a preferred embodiment, said skin exfoliating and softening composition comprises sodium polyacrylate powder at between 35% to 40% by weight.

Preferably, said base oil comprises any suitable food grade oils, synthetic, vegetable or mineral oils. Further preferably, said base oil is selected from a group consisting of avocado oil, wheatgerm oil and grapeseed oil.

Preferably, said composition may further comprise least one additive selected from: perfumes, colours, anti-settling agents, preservatives, vitamins and skin conditioning agents. Further preferably, said anti-settling agent comprises fumed silica.

Also according to the present invention there is provided a method of exfoliating and softening skin, comprising the steps of:

applying a composition comprising sodium polyacrylate powder at between 5% to 70% by weight dispersed in a solution of base oil to the skin;

rubbing said composition into the skin for a period of time to produce an exfoliating effect; and

applying water to the skin and rubbing until all of said composition is either washed away or absorbed into the skin.

It will be appreciated that the step of applying water to the skin causes said sodium polyacrylate powder to swell and releases said base oil into the surface of the skin which moisturises the surface of the skin.

Furthermore, the present invention also provides a method of manufacturing a skin exfoliating and softening composition, comprising the steps of:

combining a blend of base oils at around 28% to 93% by weight;

gradually adding an anti-settling agent at around 2% by weight under high shear; and

adding sodium polyacrylate powder at between 5% to 70% by weight to said mixture under moderate stirring.

Preferably, the method of manufacturing a skin exfoliating and softening composition further comprises the step of adding colourants at between 0.9% to 1.3% by weight under moderate stirring.

It is believed that compositions that are useful for exfoliating and softening skin in accordance with the present invention at least address the problems outlined above. The advantages of the preset invention are that compositions that are useful for exfoliating and softening skin are provided which are particularly suitable as cosmetic, toiletry, spa treatments and beauty products. Advantageously, in use, a small quantity of the composition is applied to the skin to produce an exfoliating and smoothing effect. When the skin is then rinsed in water, the sodium polyacrylate powder swells and turns to a soft gel which allows the base oil to be released into the skin which softens and moisturises the skin. Advantageously, the base oil and other additives soften and moisturise even the roughest of skin types and produces a soft exfoliating feeling on the skin. The present invention therefore provides compositions that both exfoliate and soften the skin in a single treatment. In a further advantageous aspect of the present invention, the composition opens and has a cleansing effect on skin pores.

It will be obvious to those skilled in the art that variations of the present invention are possible and it is intended that the present invention may be used other than as specifically described herein.

A specific non-limiting embodiment of the invention will now be described by way of example and with reference to the accompanying drawings, in which:

FIG. 1 shows a perspective view of the product of the present invention prior to use;

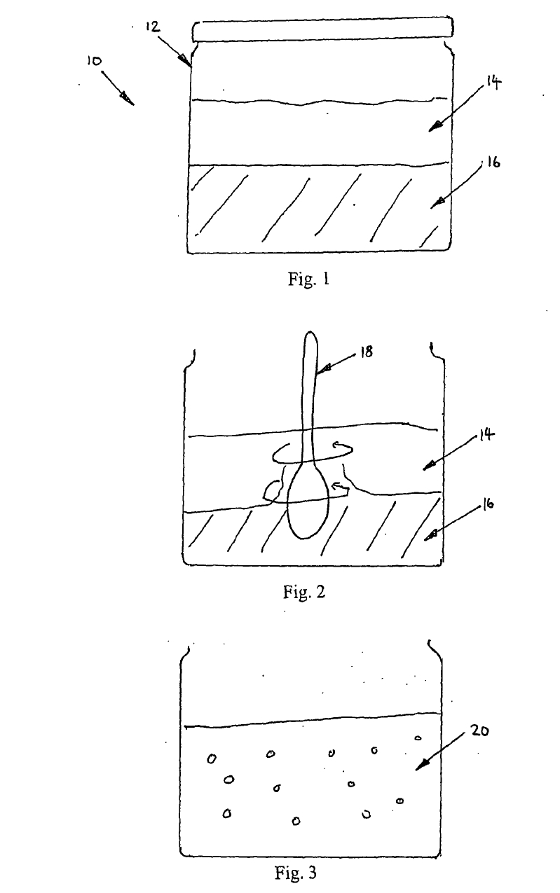

FIG. 2 illustrates how using a stirrer the product may be agitated until a free-flowing homogenous solution is formed;

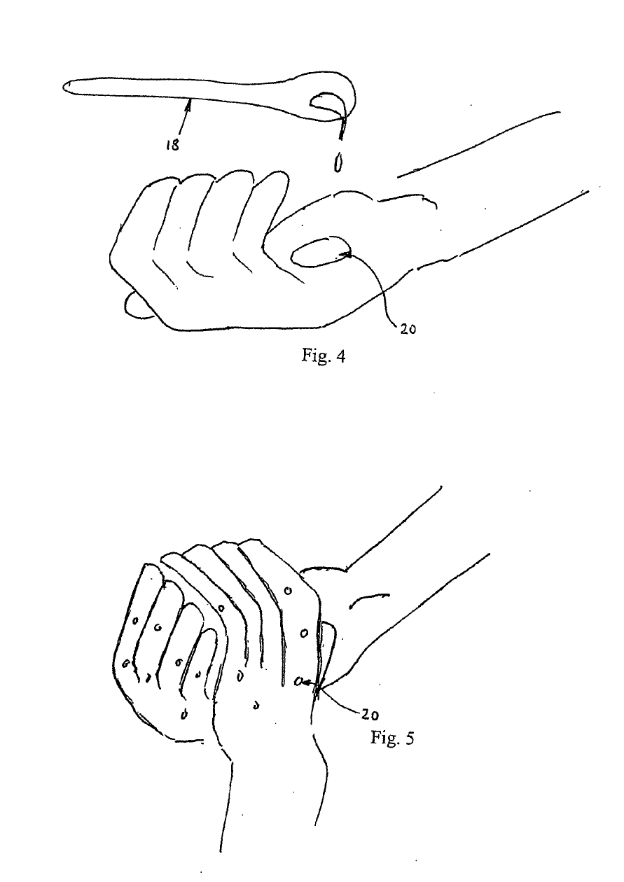

FIG. 3 shows the product having a uniform consistency.

FIG. 4 illustrates how the composition can be applied into the palm of the hands of the user.

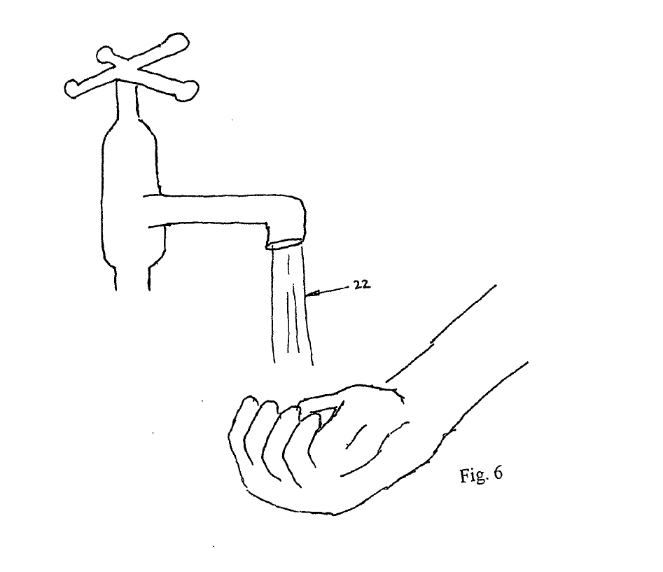

FIG. 5 shows use of the composition on the hands.

FIG. 6 illustrates the final stage of the treatment in which the hands are rinsed in water.

Referring now to the drawings, the use of the skin exfoliating and softening composition according to the present invention is shown in FIGS. 1 to 6. In particular, as shown in FIG. 1, the product 10 is sold in packaging 12. Even though the composition includes anti-settling agents, the product tends to separate out over time into a base oil phase 14 and sodium polyacrylate powder 16. FIGS. 2 and 3 show how a stirrer, such as a spoon 18 or another suitable instrument, can be used to obtain a free-flowing viscous composition 20 having a uniform consistency. As shown in FIG. 4, a small quantity of the composition 20 is placed into the palm of the hand using the spoon 18 or other measuring implement.

Although FIG. 5 depicts the use of the product 10 for exfoliating and cleansing the hands and nails, it can of course be used on any part of the body by rubbing it over the skin to produce an exfoliating and softening effect. After rubbing the skin for a short period of time, preferably two minutes, the treated area is then placed under running water 22, as shown in FIG. 6, or sprayed using an appropriate atomiser (not shown). The sodium polyacrylate powder 16 then swells and releases the base oil 14 into the skin which now moisturises the surface of the skin. At this point, the user can simply keep rubbing until all of the sodium polyacrylate powder 16 and base oil 14 is either washed away or absorbed into the skin. In use, the product 10 has exfoliating properties by firstly removing dead skin cells from the surface of the skin and deep cleansing properties by causing the skin pores to open and sucking out moisture from the skin taking with it dirt etc. This then allows the base oil 14 and cleaner moisture to be absorbed back into the skin leaving it feeling clean, smooth and soft. After this stage, the treated area is dried as normal.

The chemical composition of the invention will now be described. As shown in Tables 1 to 5, the present invention can be implemented using various base oils and other constituents. However, the invention should not be considered as being limited to the details thereof.

The composition shown in Table 1 is a fragrance-free product in which the sodium polyacrylate powder it utilised at 37.5% by weight dispersed in a solution of base oil, comprising a blend of avocado oil, wheatgerm oil and grapeseed oil. Watersorb PIFL 2024 is a brand name for a cosmetics grade sodium polyacrylate powder, although other generally available commercial products could of course be utilised. Aerosil®972 is trade name for a fumed silica which is used as an anti-settling agent or dispersant for the sodium polyacrylate powder particles in the base oil phase.

TABLE 1

| Phase | Ingredient | % w/w |
|---|---|---|
| A 1 | Ethyl Panthenol | 0.2 |
| A 2 | Tocopherol | 0.1 |
| A 3 | Avocado Oil | 1 |
| A 4 | Wheatgerm Oil | 1 |
| A 5 | α-Bisabolol | 0.05 |
| A 6 | Grapeseed Oil | 58.15 |
| B 7 | Aerosil ® 972 | 2 |
| C 8 | Watersorb PIFL 2024 | 37.5 |

The compositions shown in Tables 1 to 5 also include the constituents panthenol, tocopherol and α-Bisabolol which are particularly beneficial to the skin, since panthenol is the alcohol form of pantothenic acid, more familiar as Vitamin B5. In a living cell, panthenol is converted to pantothenic acid, which then becomes an important part of the compound ‘Coenzyme A’, which is important in cellular metabolism.

Vitamin E is an essential fat-soluble vitamin that includes eight naturally occurring compounds in two classes designated as tocopherlos and tocotrienols, as shown in Tables 1 to 5. Vitamin E has earned itself a reputation from spicing up your love life to banning wrinkles and old age. One of the most important functions of this vitamin is its antioxidant properties. Vitamin E is an effective chain-breaking, lipid-soluble antioxidant in biological membranes, and aids in membrane stability.

α-Bisabolol is a clear, colourless to slightly yellow liquid with a faint, floral, sweetish odour. It has been used for hundreds of years in medicinal applications and this naturally occurring ingredient is used to help accelerate the healing process of the skin, and can be used confidently on sensitive skins. It has anti-irritant and anti-inflammatory properties, and it also contains anti-bacterial, as well as anti-mycotic, properties.

Tables 2 and 3 show further variants of the fragrance-free composition of Table 1, and which further include various colourants and fragrances. The preferred embodiment set forth in any of Tables 1 to 3 are particularly suitable as an exfoliating and softening composition for the hands or feet or indeed anywhere where the skin requires dead skin cells to be removed. The treatment using these compositions being particularly useful prior to a manicure or false nails being applied, as the nails and skin are scrubbed prior to treatment leaving them clean, moisturised and free of other contaminants.

TABLE 2

| Phase | Ingredient | % w/w |
|---|---|---|
| A 1 | Ethyl Panthenol | 0.2 |
| A 2 | Tocopherol | 0.1 |
| A 3 | Avocado Oil | 1 |
| A 4 | Wheatgerm Oil | 1 |
| A 5 | α-Bisabolol | 0.05 |
| A 6 | Grapeseed Oil | 56.067 |
| A 7 | Perfume PN 774.016 Kiwi & Lime | 0.8 |
| B 8 | Aerosil ® 972 | 2 |
| C 9 | Watersorb PIFL 2024 | 37.5 |
| D 10 | Yellow 11 (CI 47000), 0.1% solution in oil | 1.074 |
| D 11 | Green 6 (CI 61565), 0.1% solution in oil | 0.209 |

TABLE 3

| Phase | Ingredient | % w/w |
|---|---|---|
| A 1 | Ethyl Panthenol | 0.2 |
| A 2 | Tocopherol | 0.1 |
| A 3 | Avocado Oil | 1 |
| A 4 | Wheatgerm Oil | 1 |
| A 5 | α-Bisabolol | 0.05 |
| A 6 | Grapeseed Oil | 56.42 |
| A 7 | Perfume PN 993.054 Mandarin & Starfruit | 0.8 |
| B 8 | Aerosil ® 972 | 2 |
| C 9 | Watersorb PIFL 2024 | 37.5 |
| D 10 | Yellow 11 (CI 47000), 0.1% solution in oil | 0.695 |
| D 11 | Red 17 (CI 26100), 0.1% solution in oil | 0.235 |

Tables 4 and 5 show various compositions wherein the skin exfoliating and softening composition comprises sodium polyacrylate powder at between 5% and 70% by weight, respectively. Clearly, the composition shown in Table 4, which shows sodium polyacrylate powder used at 5% by weight will have a substantially reduced exfoliating effect than, say, the composition used at 37.5% or 70% by weight and finds particular application as a gentle cleansing gel for very sensitive parts of the body. Conversely, the composition shown in Table 5, which discloses a composition including sodium polyacrylate powder at 70% by weight is particularly useful as an exfoliating gel for particular parts of the body where hard and dry skin is prevalent.

TABLE 4

| Phase | Ingredient | % w/w |
|---|---|---|
| A 1 | Ethyl Panthenol | 0.2 |
| A 2 | Tocopherol | 0.1 |
| A 3 | Avocado Oil | 1 |
| A 4 | Wheatgerm Oil | 1 |
| A 5 | α-Bisabolol | 0.05 |
| A 6 | Grapeseed Oil | 90.65 |
| B 7 | Aerosil ® 972 | 2 |
| C 8 | Watersorb PIFL 2024 | 5 |

TABLE 5

| Phase | Ingredient | % w/w |
|---|---|---|
| A 1 | Ethyl Panthenol | 0.2 |
| A 2 | Tocopherol | 0.1 |
| A 3 | Avocado Oil | 1 |
| A 4 | Wheatgerm Oil | 1 |
| A 5 | α-Bisabolol | 0.05 |
| A 6 | Grapeseed Oil | 25.65 |
| B 7 | Aerosil ® 972 | 2 |
| C 8 | Watersorb PIFL 2024 | 70 |

Any of the formulations shown in Tables 1 to 5 can be manufactured using a mixing vessel with either a sweep stirrer or a powerful propeller/turbo stirrer. A method of manufacturing a skin exfoliating and softening composition according to any of formulations disclosed in Tables 1 to 5 will now be described. In particular, in the first stage, the ingredients of phase A are firstly mixed together and then phase B is carefully added under fast agitation and such is stirred until completely dispersed. A high shear mixer may be helpful for this stage in the manufacturing process. After phases A and B have been combined, the high shear mixer is then turned off and phase C is then added with moderate stirring. For the formulations of set forth in Tables 2 and 3, the colourants of phase D are then finally added, again, under moderate stirring.

Various alterations or modifications may be made to the present invention without departing from the scope of the invention.

1. A skin exfoliating and softening composition, comprising sodium polyacrylate powder at between 5% to 70% by weight dispersed in a solution of base oil.

2. The skin exfoliating and softening composition according to claim 1, further comprising sodium polyacrylate powder at between 20% to 55% by weight.

3. The skin exfoliating and softening composition according to claim 1, further comprising sodium polyacrylate powder at between 35% to 40% by weight.

4. The skin exfoliating and softening composition according to claim 1, wherein said base oil comprises any suitable food grade oils, synthetic, vegetable or mineral oils.

5. The skin exfoliating and softening composition according to claim 1, wherein said base oil is selected from the group consisting of avocado oil, wheatgerm oil and grapeseed oil.

6. The skin exfoliating and softening composition according to claim 1, further comprising at least one additive selected from the group consisting of perfumes, colours, anti-settling agents, preservatives, vitamins and skin conditioning agents.

7. The skin exfoliating and softening composition according to claim 6, wherein said anti-settling agent comprises fumed silica.

8. A method of exfoliating and softening skin, comprising the steps of:
applying a composition comprising sodium polyacrylate powder at between 5% to 70% by weight dispersed in a solution of base oil to the skin;
rubbing said composition into the skin for a period of time to produce an exfoliating effect; and
applying water to the skin and rubbing until all of said composition is either washed away or absorbed into the skin.

9. The method of exfoliating and softening skin according to claim 8, wherein the step of applying water to the skin causes said sodium polyacrylate powder to swell and releases said base oil into the surface of the skin which moisturises the surface of the skin.

10. A method of manufacturing a skin exfoliating and softening composition, comprising the steps of:
combining a blend of base oils at around 28% to 93% by weight;
gradually adding an anti-settling agent at around 2% by weight under high shear; and
adding sodium polyacrylate powder at between 5% to 70% by weight to said mixture under moderate stirring.

11. The method of manufacturing a skin exfoliating and softening composition according to claim 9, further comprising the step of adding colourants at between 0.9% to 1.3% by weight under moderate stirring.

12. (canceled)

13. (canceled)

14. (canceled)
